(19) United States
(12) Patent Application Publication
(10) Pub. No.: US 2026/0279500 A1
(43) Pub. Date: Sep. 17, 2026

(54) SYSTEMS AND METHODS FOR ENDPOINT GENOTYPING

(71) Applicants: Roche Molecular Systems Inc., Pleasanton, CA (US); Roche Sequencing Solutions, Inc., Pleasanton, CA (US)

(72) Inventors: Corey Hoeppner, Foster City, CA (US); Ronald Kurnik, Dublin, CA (US)

(21) Appl. No.: 19/675,984

(22) Filed: May 13, 2026

Related U.S. Application Data
(63) Continuation of application No. PCT/EP2024/082015, filed on Nov. 12, 2024.
(60) Provisional application No. 63/598,258, filed on Nov. 13, 2023.

(51) Int. Cl. G16B 40/10; G16B 20/00; G16B 45/00

(52) U.S. Cl. CPC G16B 40/10; G16B 20/00; G16B 45/00

(57) ABSTRACT

The present disclosure relates to performing endpoint genotyping based on a Gaussian mixture model (GMM). As one example, a method includes: obtaining, using a processor, cross-talk corrected fluorescent PCR data for an assay; determining, using the processor, a plurality of values based on the cross-talk corrected fluorescent PCR data; generating, using the processor, an assay based on the plurality of values; determining, using the processor, a genotype of a sample based on one or more gray-zones and at least one value associated with the sample; and displaying the genotype determination on a user display.

### CROSS-REFERENCE TO RELATED APPLICATIONS

The present application is a continuation of International Patent Application No. PCT/EP2024/082015, filed Nov. 12, 2024, which application claims benefit of priority to U.S. Provisional Application No. 63/598,258, filed Nov. 13, 2023, each of which is incorporated by reference herein in its entirety.

### TECHNICAL FIELD

The present disclosure relates to methods for endpoint genotyping, and more particularly, to methods for performing endpoint genotyping based on a Gaussian mixture model (GMM).

### BACKGROUND

The polymerase chain reaction (PCR) has become a ubiquitous tool of biomedical research, disease monitoring, and diagnostics. Using PCR data, genotyping may be used to detect genetic differences. More specifically, genotyping includes comparing a DNA sequence to that of another sample or a reference sequence in order to determine differences in genetic complement, and it is used to identify small variations in genetic sequences within populations, such as single-nucleotide polymorphisms (SNPs).

One example type of genotyping is endpoint genotyping, which uses two different probes that each bind to a target, e.g., an allele for a specific gene of interest. Each probe can be incorporated into an amplicon containing their specific allele, while simultaneously releasing fluorescence at their specific wavelength. Ratios of fluorescence from these probes is then analyzed to determine which allele the probes were binding to. These ratios can be used to perform endpoint genotyping to determine whether a sample is a wild type, mutant, or heterozygote.

### SUMMARY

The present disclosure provides for novel methods for performing endpoint genotyping based on a Gaussian mixture model (GMM). As one example, a method includes obtaining, using a processor, cross-talk corrected fluorescent PCR data for an assay. The method may further include determining, using the processor, a plurality of values based on the cross-talk corrected fluorescent PCR data. The method may further include generating, using the processor, an assay based on the plurality of values. The method may further include determining, using the processor, a genotype of a sample based on one or more gray-zones and at least one value associated with the sample. The method may further include displaying the genotype determination on a user display.

According to some aspects, the determining the plurality of values includes: determining first values for a pair of channels that indicates a deviation from a linear approximation of fluorescence values that is fit to the real-time PCR data from a plurality of cycles of an analyzer; determining a second value based on the first values of each channel of the pair of channels; and determining a third value based on a plurality of cycle thresholds of a target species.

According to some aspects, the second value is based on a log ratio of the first value of each channel of the pair of channels and the third value comprises a mean value based on the plurality of cycle thresholds.

According to some aspects, the generating the assay comprises generating a Gaussian mixture model (GMM).

According to some aspects, the GMM comprises a plurality of Gaussians mixtures with a number of Gaussians mixtures corresponding to a number of genotypes.

According to some aspects, the generating the assay comprises determining mean values of the plurality of Gaussians mixtures.

According to some aspects, the determining the mean values of the plurality of Gaussian mixtures comprises determining the mean values of the plurality of Gaussians mixtures using an expectation maximization technique.

According to some aspects, the generating the assay comprises determining at least one threshold value based on the mean values of the plurality of Gaussians mixtures.

According to some aspects, the generating the assay comprises determining the at least one gray-zone based on the at least one threshold value.

According to some aspects, the determining the genotype comprises comparing the value associated with the sample to the one or more gray-zones.

According to some aspects, the determining the genotype comprises determining a quality metric score.

According to some aspects, the displaying the genotype comprises displaying the genotype using a scatter plot.

### BRIEF DESCRIPTION OF THE DRAWINGS

FIG. 1 is a block diagram illustrating an embodiment an optical system of an analyzer, according to aspects of the present disclosure.

FIG. 2 shows a method for classifying unknown samples into known genotypes, according to aspects of the present disclosure.

FIG. 3 is a block diagram of a computing system in accordance with embodiments of the present disclosure.

FIG. 4 illustrates two example fluorescence curves in accordance with embodiments of the present disclosure.

FIG. 5 illustrates a deviation from a linear approximation of the fluorescence curves in accordance with embodiments of the present disclosure.

FIGS. 6 and 7 illustrate example scatter plots in accordance with embodiments of the present disclosure.

FIGS. 8 and 9 illustrate example scatter plots with one or more gray-zones in accordance with embodiments of the present disclosure.

### DETAILED DESCRIPTION

In the context of in vitro diagnostic (IVD) assays, it is desirable to determine different genotypes. To that end, and as described above, PCR data may be used in endpoint genotyping to identify genetic mutations. The present disclosure provides for novel methods for performing endpoint genotyping based on a Gaussian mixture model (GMM).

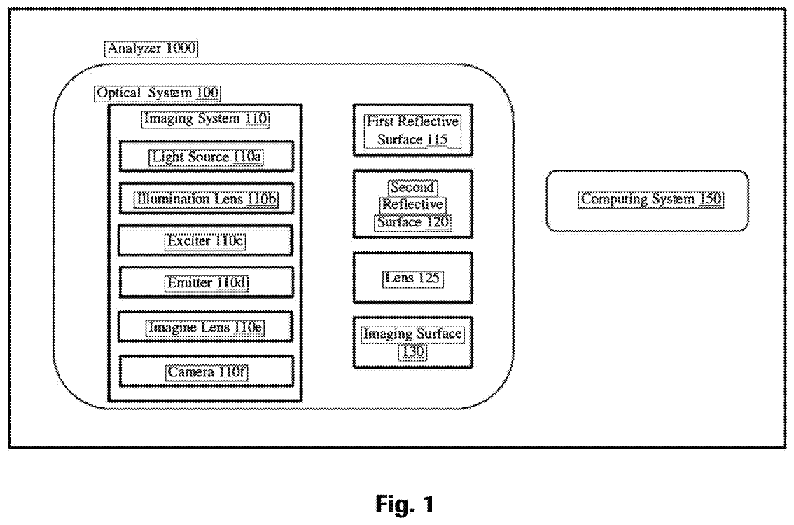

FIG. 1 is a block diagram illustrating an embodiment of an analyzer, according to aspects of the present disclosure. For example, as shown in FIG. 1, an analyzer 1000 includes an optical system 100 and a computing system 150. In some embodiments, the optical system 100 includes an imaging system 110, first and second reflective surfaces 115 and 120, respectively, a lens 125, and an imaging surface 130. In some embodiments, the imaging system 110 may include a light source 110a configured to generate a beam of light, an illumination lens 110b configured to focus the beam of light, and an exciter 110c configured to transmit the focused beam of light onto the first reflective surface 115. In some embodiments, the focused beam of light is reflected off the first reflective surface 115 onto the second reflective surface 120, which in turn is transmitted onto the imaging surface 130 through the lens 125.

In turn, light is reflected from the imagining surface 130 through lens 125 and off of the first and second reflective surfaces 115, 120 onto the imaging system 110. In some embodiments, the imaging system 110 may further include an emitter 110d configured to receive the reflected light from the imaging surface 130, an imaging lens 110e configured to focus the reflected light, and a camera 110f configured to capture the reflected light from the imaging surface 130. In some embodiments, the camera 110f can be used for fluorescence imaging due to its high sensitivity, low noise, and high temporal stability.

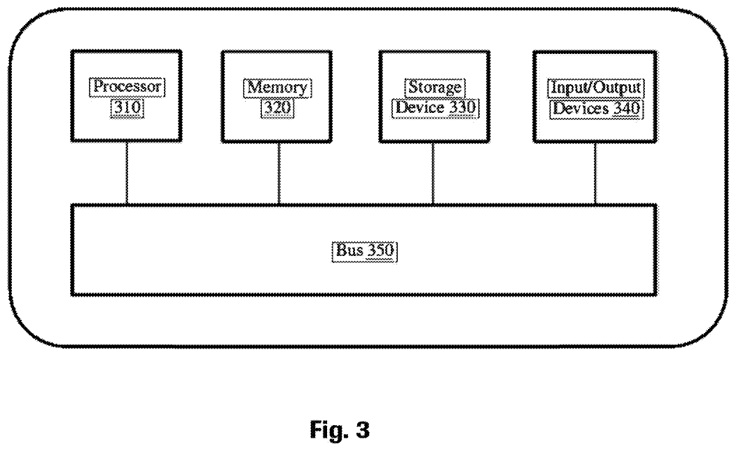

In some embodiments, the computing system 150 may execute one or more processes for performing endpoint genotyping. An example architecture of the computing system 150 is shown in FIG. 3, discussed in greater detail below.

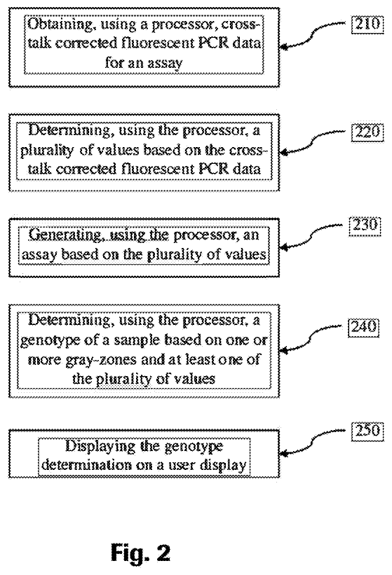

FIG. 2 illustrates a method 200 for performing endpoint genotyping. In some embodiments, the processes described herein may be performed using a processor, e.g., processor 310 of FIG. 3. At step 210, the method 200 includes obtaining, using the processor 310, cross-talk corrected fluorescent PCR data for an assay, where genotypes are distinguished in two channels. In some embodiments, the assay may be any assay used by an analyzer, e.g., the analyzer 1000 of FIG. 1, which uses real-time polymerase chain reaction (PCR) for the detection and genotyping of the human mutations. For example, the assays may include assays for detecting Factor II (FII) and Factor V (FV) alleles. Furthermore, in some embodiments, the cross-talk corrected fluorescent PCR data may be obtained for a plurality of different channels using a respective one of a plurality of probes, e.g., a first probe may be used in a first channel, a second probe may be used in a different channel, etc. In some embodiments, different genotypes may be determined using pairs of channels from among the plurality of channels. For example, Factor V genotypes may be determined using a pair of channels and Factor II genotypes may be determined using a second pair of channels, where the first and second pairs of channels are mutually exclusive.

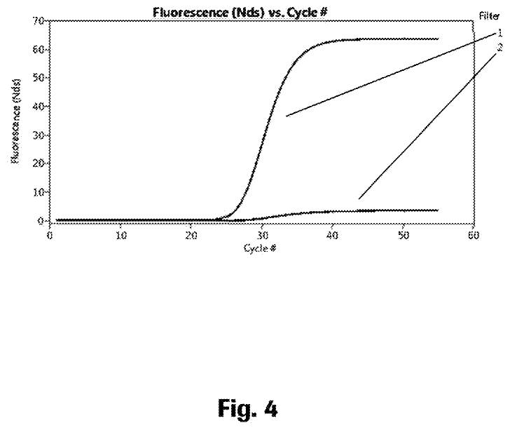

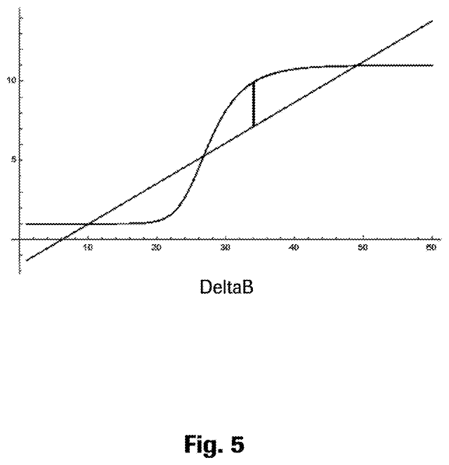

At step 220, the method 200 includes determining, using the processor 310, a plurality of values based on the cross-talk corrected fluorescent PCR data. For example, the determining the plurality of includes determining a first value DeltaB for a plurality of channels. FIG. 4 illustrates two example fluorescence curves for Factor V in the first pair of channels, e.g., a first channel and a second channel from among the plurality of channels. In some embodiments, as shown in FIG. 5, the first value DeltaB indicates a deviation from a linear approximation of the fluorescence values that is fit to the real-time PCR data from a plurality of cycles of the analyzer 1000, e.g., from a sixth cycle to a last cycle. The linear approximation may be determined using a straight line ŷ=a+bx, where values a and b may be estimated using a linear least-squares technique, as should be understood by those of ordinary skill in the art, and the value y represents the actual fluorescent data. In accordance with aspects of the present disclosure, the first value DeltaB may be determined using equation (1):

$$DeltaB = \frac{max \vert {\hat{y}}_{i} - {y}_{i} \vert}{y M e d i a n} \qquad ( 1 )$$

In some embodiments, yMedian may be determined by sorting the fluorescence data in ascending order and taking the median of the first five (5) data points of the sorted list. In some embodiments, when yMedian is ≤10⁻², then yMedian is set to a value of 0.01.

In some embodiments, the first value may be based a cycle range of the analyzer 1000, e.g., initial cycle number and final cycle number, and truncation by decline detection. For example, when an initial cycle number is give, then the linear fit calculation and the median calculation may be performed after six cycle, e.g., starting at cycle 11. Furthermore, in some implementations, the use x-values of 11, . . . , last cycle when performing this linear regression.

In some embodiments, the first value may be set to zero when a normalization value norm is less than 0.001. The normalization value norm may be determined as follows

$$norm = \sqrt{{\sum }_{start}^{stop} {y}_{i}^{2}} ,$$

where yᵢ=fluorescent values and start and stop are the cycle range initial and final.

In some embodiments, the determining the plurality of values may further include determining a second value based on the first values of each channel of the pair of channels. For example, the second value may be a log ratio LDBR of the first value of each channel of the pair of channels. The log ratio LDBR may be, for example, a common logarithmic function.

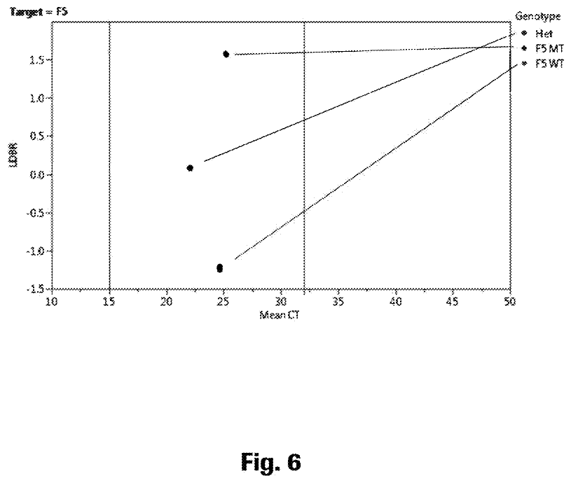

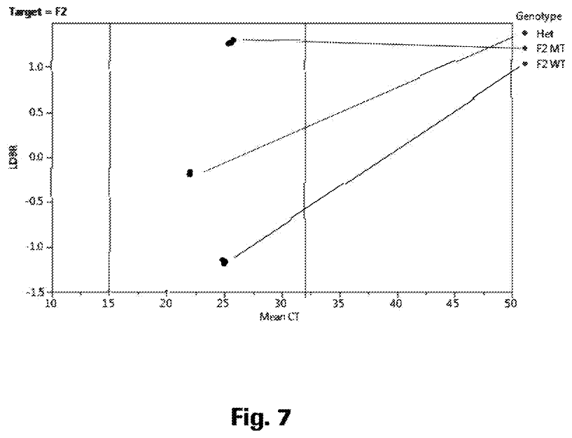

In some embodiments, the determining the plurality of values may further include determining a third value based on a plurality of cycle thresholds of a target species. For example, the third value may be a mean value of the plurality of cycle thresholds of the target species. In some embodiments, these calculations may be repeated for all test data and summarized in a scatter plot as shown in FIGS. 6 and 7, respectively. As shown in the examples of FIGS. 6 and 7, the LDBR plots may be used to cluster the values into one of the three possible genotypes, e.g., wild type, mutant, or heterozygote, for Factor V genotypes and Factor II genotypes, respectively.

At step 230, the method 200 may further include generating, using the processor 310, an assay based on the plurality of values. For example, generating the assay may include generating a Gaussian mixture model (GMM). In some embodiments, the GMM may include a plurality of Gaussians mixtures with a number of Gaussians mixtures corresponding to a number of genotypes. For example, the GMM may include three Gaussians mixtures to identify three different genotypes or two Gaussians mixtures to identify two different genotypes.

In some embodiments, the generating the assay may include determining mean values of the plurality of Gaussians mixtures. For example, the mean values of the plurality of Gaussians mixtures may be determined using an expectation maximization technique. In the following example, K is equal to three (3), representing the three Gaussian mixtures. In some embodiments, a probability p(x) that the log ratio LDBR is located in a given Gaussian mixture is determined using:

$$p ( x ) = {\sum }_{i = 1}^{K} {ϕ}_{i} N ( x \vert {μ}_{i} , {σ}_{i} ) , \qquad ( 2 )$$

where φᵢ is the fraction of a given Gaussian mixture of the Gaussian sum and the label N represents the normal distribution, as shown in:

$$N ( x \vert {μ}_{i} , {σ}_{i} ) = \frac{1}{{σ}_{i} \sqrt{2 π}} exp ( - \frac{{( x - {μ}_{i} )}^{2}}{2 {σ}_{i}^{2}} ) \qquad ( 3 )$$

According to aspects of the present disclosure, the sum of the plurality of Gaussian mixtures must equal one (1) as shown in:

$${\sum }_{i = 1}^{K} {ϕ}_{i} = 1. \qquad ( 4 )$$

In some embodiments, the expectation maximization technique may include determining initial mean values of the plurality of Gaussian mixtures. For example, for three Gaussian mixtures, initial mean values μᵢ of the plurality of Gaussian mixtures may be chosen as μ₁=minimum LDBR value and μ₃=maximum LDBR value from a training data set, while μ₂=(μ₁+μ₃)/2. In contrast, for two Gaussian mixtures, the initial mean values μᵢ may be chosen as μ₁=minimum LDBR value and μ₂=maximum LDBR value from a training data set. In some embodiments, an initial value of the standard deviation σᵢ may be fixed at 0.1 across the plurality of Gaussian mixtures.

In some embodiments, the expectation maximization technique may also include determining a provisional expectation value indicating that a given log ratio LDBR is in a Gaussian mixture. The expectation value may be determined using:

$${\hat{γ}}_{ik} = \frac{{\hat{ϕ}}_{k} N ( {x}_{i} \vert {\hat{μ}}_{k} , {\hat{σ}}_{k} )}{{{\sum }_{{j = 1}^{}}}^{K} {\hat{ϕ}}_{j} N ( {x}_{i} \vert {\hat{μ}}_{j} , {\hat{σ}}_{j} )} . \qquad ( 5 )$$

In some embodiments, the expectation maximization technique may further include determining updated provisional values of φᵢ, μᵢ, σᵢ based on the provisional expectation. The updated provisional values of φᵢ, μᵢ, σᵢ may be determined using, and Error! Reference source not found.:

$${\hat{ϕ}}_{k} = {\sum }_{i = 1}^{N} \frac{{\hat{γ}}_{i k}}{N} \qquad Equation 6$$

$${\hat{μ}}_{k} = \frac{{\sum }_{i = 1}^{N} {\hat{γ}}_{i k} {x}_{i}}{{\sum }_{i = 1}^{N} {\hat{γ}}_{i k}} \qquad Equation 7$$

$${\hat{σ}}_{k}^{2} = \frac{{\sum }_{i = 1}^{N} {( \hat{γ} )}_{ik} {( {x}_{i} - {\hat{μ}}_{k} )}^{2}}{{\sum }_{i = 1}^{N} {\hat{γ}}_{ik}} \qquad Equation 8$$

In some embodiments, the determining the provisional expectation value and the determining the updated provisional values of φᵢ, μᵢ, σᵢ based on the provisional expectation may be executed in a loop with a maximum number of iterations. For example, the maximum number of iterations may be twenty-five (25). In some embodiments, the loop may be stopped before the maximum number of iterations based on Equation 9:

$${\sum }_{j = 1}^{K} \vert {μ}_{i , j} - {μ}_{i - 1 , j} \vert \leq 0.00005 \qquad ( 9 )$$

In some embodiments, the generating the assay may include determining at least one threshold value based on the mean value(s). For example, for three genotypes, the at least one threshold may include two threshold values, and the threshold values may be determined using equations 10 and 11, respectively:

$$Threshold 1 = \frac{{μ}_{1} + {μ}_{2}}{2} \qquad ( 10 )$$

$$Threshold 2 = \frac{{μ}_{2} + {μ}_{3}}{2} \qquad ( 11 )$$

In some embodiments, the generating the assay may include determining at least one gray-zone based on the at least one threshold value. In some embodiments, a number of gray-zones corresponds to the number of threshold values. For example, for two threshold values, two corresponding gray-zones may. In some embodiments, the gray-zones may be determined using equations 12 and 13:

$$GZ 1 = Threshold 1 \pm \vert {μ}_{1} - {μ}_{2} \vert * 0 \text{.05} \qquad ( 12 )$$

$$GZ 2 = Threshold 2 \pm \vert {μ}_{2} - {μ}_{3} \vert * 0.05 . \qquad ( 13 )$$

It should be understood by those of ordinary skill in the art that the number of threshold values and gray-zones corresponds to the number of endpoint genotypes. For example, for a two endpoint genotypes, a single threshold value and a single corresponding gray-zone are used.

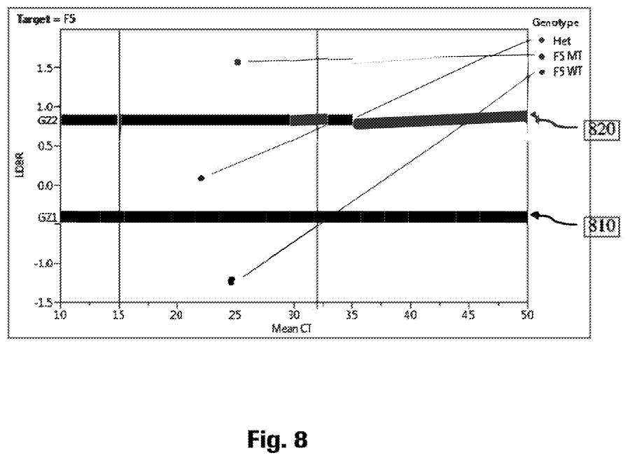

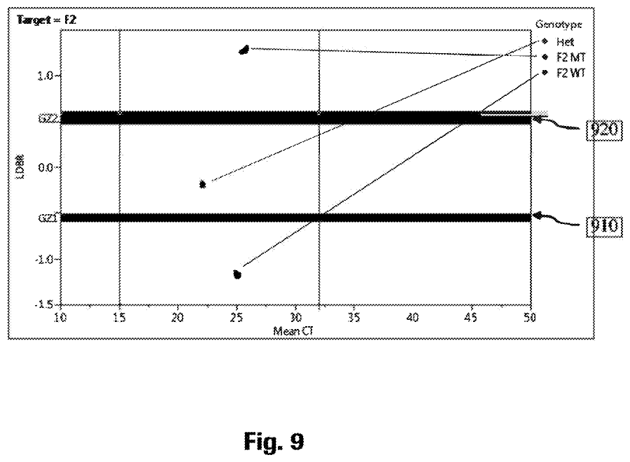

In some embodiments, at step 240, the method 200 may further include determining, using the processor 310, a genotype of a sample based on the one or more gray-zones and at least one of the plurality of values. For example, the second value of the sample obtained during a PCR process may be compared to each gray zone to determine a genotype. In some embodiments, the at least one value of the sample may be the second value of the plurality of values, as discussed herein. Using three genotypes as an example, as shown in FIGS. 8 and 9, the sample may be identified as a first genotype when the second value is less than a value defined by a first gray-zone 810, 910, as a second genotype when the second value is greater than the value defined by a first gray-zone 810, 910 and less than a value defined by a second gray-zone 820, 920, or as a third genotype when the second value is greater than the value defined by the second gray-zone 820, 920.

In some embodiments, the determining the genotype may include determining a quality metric score. The quality metric score may indicate a confidence in categorizing the samples a given genotype. For example, for a first genotype, the quality metric score may be determined using equation 14:

$$Quality Metric Score = Min ( \frac{L D B R - {μ}_{2}}{{μ}_{3} - {μ}_{2}} , 1 ) * 100. \qquad ( 14 )$$

For example, for a second genotype, the quality metric score may be determined using equation 15:

$$Score = Min ( \frac{{μ}_{2} - L D B R}{{μ}_{2} - {μ}_{1}} , 1 ) * 100. \qquad Equation ( 15 )$$

For example, for a third genotype, the quality metric score may be determined using equation 16:

$$Score = Min ( \frac{L D B R - {μ}_{1}}{{μ}_{2} - {μ}_{1}} , \frac{{μ}_{3} - L D B R}{{μ}_{3} - {μ}_{2}} ) * 100. \qquad ( 16 )$$

At step 250, the method 200 may further include displaying the genotype determination on a user display, e.g., input/output devices 340 of FIG. 3. In some embodiments, the displaying the genotype may include displaying the genotype using a scatter plot, as shown in FIGS. 8 and 9, respectively.

FIG. 3 depicts a block diagram illustrating an example of computing system 300, in accordance with some example embodiments. In some embodiments, the computing system 300 may be to implement the method 200 and/or any components therein.

As shown in FIG. 3, computing system 300 can include a processor 310, a memory 320, a storage device 330, and input/output devices 340. Processor 310, memory 320, storage device 330, and input/output devices 340 can be interconnected via system bus 350. Processor 310 is capable of processing instructions for execution within the computing system 300. Such executed instructions can implement one or more components of, for example, analyzer 1000, method 200 and/or any components therein. In some example embodiments, processor 310 can be a single-threaded processor. Alternately, processor 310 can be a multi-threaded processor. Processor 310 is capable of processing instructions stored in memory 320 and/or on the storage device 330 to display graphical information for a user interface provided via the input/output device 340.

Memory 320 is a computer readable medium such as volatile or non-volatile that stores information within computing system 300. Memory 320 can store data structures representing configuration object databases, for example. Storage device 330 is capable of providing persistent storage for computing system 300. Storage device 330 can be a floppy disk device, a hard disk device, an optical disk device, or a tape device, or other suitable persistent storage means. Input/output device 340 provides input/output operations for the computing system 300. In some example embodiments, input/output device 340 includes a keyboard and/or pointing device. In various implementations, the input/output device 340 includes a display unit for displaying graphical user interfaces.

According to some example embodiments, input/output device 340 can provide input/output operations for a network device. For example, input/output device 340 can include Ethernet ports or other networking ports to communicate with one or more wired and/or wireless networks (e.g., a local area network (LAN), a wide area network (WAN), the Internet).

In some example embodiments, computing system 300 can be used to execute various interactive computer software applications that can be used for organization, analysis and/or storage of data in various formats. Alternatively, computing system 300 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via input/output device 340. The user interface can be generated and presented to a user by computing system 300 (e.g., on a computer screen monitor, etc.).

One or more aspects or features of the subject matter described herein can be realized in digital electronic circuitry, integrated circuitry, specially designed application-specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

Embodiments of the present invention will be further described in the following examples, which do not limit the scope of the invention described in the claims.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A method comprising:
obtaining, using a processor, cross-talk corrected fluorescent PCR data for an assay;
determining, using the processor, a plurality of values based on the cross-talk corrected fluorescent PCR data;
generating, using the processor, an assay based on the plurality of values;
determining, using the processor, a genotype of a sample based on one or more gray-zones and at least one value associated with the sample; and
displaying the genotype determination on a user display.

2. The method of claim 1, wherein determining the plurality of values comprises:
determining first values for a pair of channels that indicates a deviation from a linear approximation of fluorescence values that is fit to the real-time PCR data from a plurality of cycles of an analyzer;
determining a second value based on the first values of each channel of the pair of channels; and
determining a third value based on a plurality of cycle thresholds of a target species.

3. The method of claim 2, wherein:
the second value is based on a log ratio of the first value of each channel of the pair of channels; and
the third value is a mean value based on the plurality of cycle thresholds.

4. The method of claim 1, wherein the generating the assay comprises generating a Gaussian mixture model (GMM).

5. The method of claim 4, wherein the GMM comprises a plurality of Gaussians mixtures with a number of Gaussians mixtures corresponding to a number of genotypes.

6. The method of claim 4, wherein the generating the assay comprises determining mean values of the plurality of Gaussians mixtures.

7. The method of claim 6, wherein the determining the mean values of the plurality of Gaussian mixtures comprises determining the mean values of the plurality of Gaussians mixtures using an expectation maximization technique.

8. The method of claim 6, wherein the generating the assay comprises determining at least one threshold value based on the mean values of the plurality of Gaussians mixtures.

9. The method of claim 8, wherein the generating the assay comprises determining the at least one gray-zone based on the at least one threshold value.

10. The method of claim 1, wherein the determining the genotype comprises comparing the value associated with sample to the one or more gray-zones.

11. The method of claim 1, wherein the determining the genotype comprises determining a quality metric score.

12. The method of claim 1, wherein the displaying the genotype comprises displaying the genotype using a scatter plot.

13. A system comprising:
a memory; and
a processor coupled to the memory and configured to:
obtain cross-talk corrected fluorescent PCR data for an assay;
determine a plurality of values based on the cross-talk corrected fluorescent PCR data;
generate an assay based on the plurality of values;
determine a genotype of a sample based on one or more gray-zones and at least one value associated with the sample; and
cause display the genotype determination on a user display.
